(19) United States
(12) Patent Application Publication
(10) Pub. No.: US 2014/0229200 A1
(43) Pub. Date: Aug. 14, 2014

(54) SYSTEMS AND METHODS FOR MANAGING AND DYNAMICALLY PREDICTING RESPIRATORY EPISODES

(71) Applicant: iSonea Limited, Armadale (AU)

(72) Inventors: Johnny Yat Ming CHAN, Newport Beach, CA (US); Stephen Anthony TUNNELL, Oceanside, CA (US); Michael Joseph THOMAS, Severna Park, MD (US)

(73) Assignee: iSonea Limited, Armadale (AU)

(21) Appl. No.: 14/180,743

(22) Filed: Feb. 14, 2014

Related U.S. Application Data
(60) Provisional application No. 61/764,864, filed on Feb. 14, 2013.

(51) Int. Cl. G06F 19/00; A61B 5/08

(52) U.S. Cl. CPC G06F 19/3431; A61B 5/08; G06F 19/3406
USPC 705/3

(57) ABSTRACT

The disclosure is directed to devices, systems, kits and methods for managing respiratory episodes and providing dynamic respiratory predictions of the likelihood of occurrence of a respiratory episode prior to potential onset of respiratory episodes based on physiological data, environmental data and patient data for other patients in the system.

### CROSS-REFERENCE

This application claims the benefit of U.S. Provisional Application No. 61/764,864 filed Feb. 14, 2013, which application is incorporated herein by reference.

### BACKGROUND OF THE INVENTION

Asthma is an episodic chronic disease that disrupts normal respiratory function in mammals. One aspect of asthma therapy involves preventing episodes of extreme worsening of respiratory function such as those associated with asthma attacks. During an asthma attack, or asthma exacerbation, the patient's airways become swollen and inflamed. Additionally, muscles associated with the patient's airways contract which causes the bronchial tubes to narrow. Patients will often wheeze, cough, and have trouble breathing. The severity of the attack can be minor or result in a life threatening emergency requiring a trip to the hospital.

The US National Institutes of Health (NIH) has recommended that asthma suffers take an Asthma Control Test (ACT) to assess the level of control of asthma. ACT is a tool that patients and healthcare providers use to assess asthma conditions and control. Patients answer a series of questions which look back over a period of time to assess whether shortness of breath was experienced, number of times a patient awoke during their sleep cycle from asthma relates symptoms (shortness of breath, chest tightness, or pain), the number of times a rescue inhaler was used (such as albuterol), as well as a subjective personal rating of the patient's impression of control over the same period of time.

Other references relating to asthma include, for example, U.S. Pat. No. 8,231,541 B2 for Asthma Status Scoring Method and System with Confidence Ratings issued Jul. 31, 2012 to Colquitt et al.

Managing respiratory episodes, such as asthma attacks is greatly assisted by recognizing and treating flare-ups early and understanding when the attacks get worse and how to deal with an attack in progress.

What is needed is a respiratory functionality system assessment and predictor and method that can determine the likelihood of a respiratory incident, such as an asthma attack, prior to its onset. Additionally what is needed is a system and method that can determine the likelihood of a respiratory incident (such as an asthma attack) and the change in the risk profile (changes for the better or changes for the worse) for the person(s) monitoring their respiratory condition that is configurable to operate as part of a communication network.

### SUMMARY OF THE INVENTION

Systems and methods that can determine the likelihood of a respiratory incident, such as an asthma attack.

An aspect of the disclosure is directed to a system comprising: an electronic device configurable to be in communication with a communication network; a computer executable instruction that, when executed by a processor determines a likelihood of a respiratory event for a user based on two or more of historical user data, current environmental data, current physiological data for one or more other users in a similar geographic location and historical data for one or more of the other user in a similar geographic location. In some configurations, one or more of the historical user data and historical data for one or more other users is accessible via the communication network. Additionally, a messaging system can be included wherein the messaging system is capable of delivering a respiratory event message to one or more of the user, one or more of the other users, and a healthcare provider, or all of them. In some configurations, the electronic device is at least one of a respiratory sensor and a mobile communication device. Moreover, the computer executable instruction is processable on one or more of the electronic device, a remote server, and an electronic device within near field communication range.

Another aspect of the disclosure is directed to a non-transitory computer readable medium storing instructions that, when executed by a computing device, causes the computing device to perform a method, the method comprising: receiving one or more of GPS location and a physiological condition indication for a first user; at least one or more of analyzing, monitoring, evaluating, and providing a respiratory prediction for a second user based on the GPS location of the second user and the physiological condition indication for the first user. In some configurations, the method includes the step of at least one or more of analyzing and evaluating one or more of a historical data for the first user and historical data for second user. Additionally, the medium can be configured such that it is capable of generating and delivering a respiratory event message to one or more of the user, one or more of the other users, and a healthcare provider. The computing device performing the method can be, for example, a respiratory sensor and a mobile communication device. Additionally, the method performed by the computer readable medium can be processed on one or more of the electronic device, a remote server, and an electronic device within near field communication range.

Yet another aspect of the disclosure is directed to a computing device comprising: a processor configured to: receive one or more of GPS location and physiological condition indication for a first user; at least one or more of analyze, monitor, evaluate, and provide a respiratory prediction for one or more second users based on the GPS location of the one or more second users and the physiological condition indication for the first user. In some configurations, the processor can perform the step of at least one or more of analyzing and evaluating one or more of a historical data for the first user and historical data for second user. Additionally, the processor can be configured such that it is capable of generating and delivering a respiratory event message to one or more of the user, one or more of the other users, and a healthcare provider. The computing device can be, for example, a respiratory sensor and a mobile communication device. Additionally, the processor can be provided on one or more of the electronic device, a remote server, and an electronic device within near field communication range.

Still another aspect of the disclosure is directed to a method comprising: receiving one or more of GPS location and a physiological condition indication for a first user; at least one or more of analyzing, monitoring, evaluating, and providing a respiratory prediction for one or more second users based on the GPS location of the one or more second users and the physiological condition indication for the first user. The method can also include the step of at least one or more of analyzing and evaluating one or more of a historical data for the first user and historical data for second user. In some configurations, the medium is capable of generating and delivering a respiratory event message to one or more of the first user, one or more of the other users, a healthcare provider and/or a central location.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a block diagram showing a representative example of a logic device through which respiratory function management and prediction can be achieved;

FIG. 1B is a block diagram of an exemplary computing environment through which respiratory function management and prediction can be achieved;

FIG. 1C is an illustrative architectural diagram showing some structure that can be employed by devices through which respiratory function management and prediction is achieved;

FIG. 2 is a block diagram showing the cooperation of exemplary components of a system suitable for use in a system where respiratory function management and prediction is achieved; and

FIG. 3 illustrates an interrelationship between data components in the system.

### DETAILED DESCRIPTION OF THE INVENTION

### I. Computing Systems

The systems and methods described herein rely on a variety of computer systems, networks and/or digital devices for operation. In order to fully appreciate how the system operates an understanding of suitable computing systems is useful. Aspects of the systems and methods disclosed herein can be enabled as a result of application via a suitable computing system.

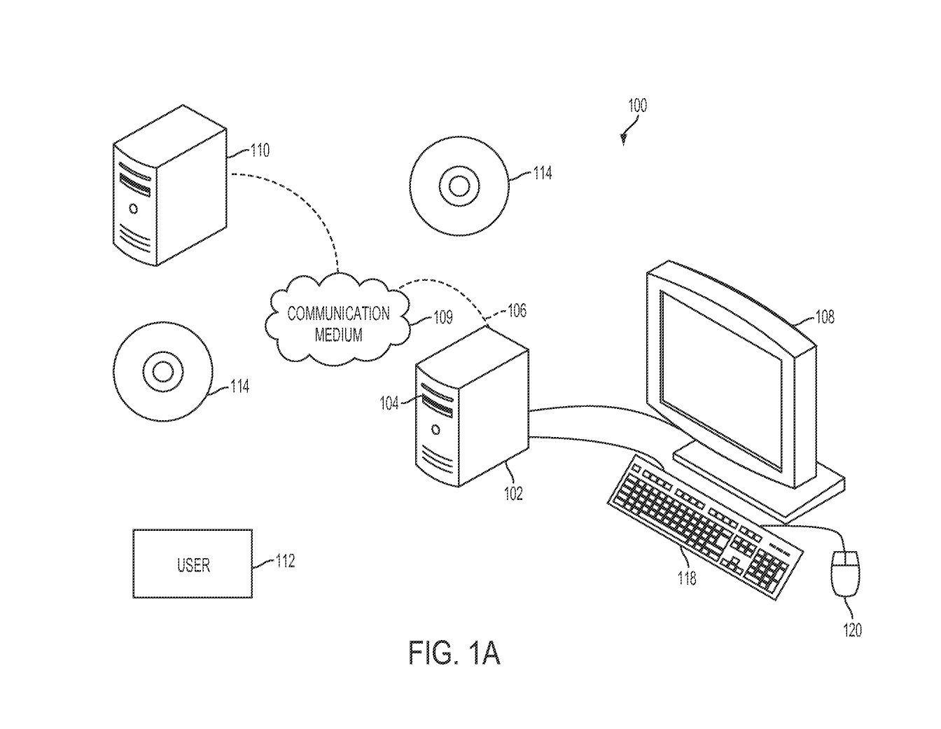

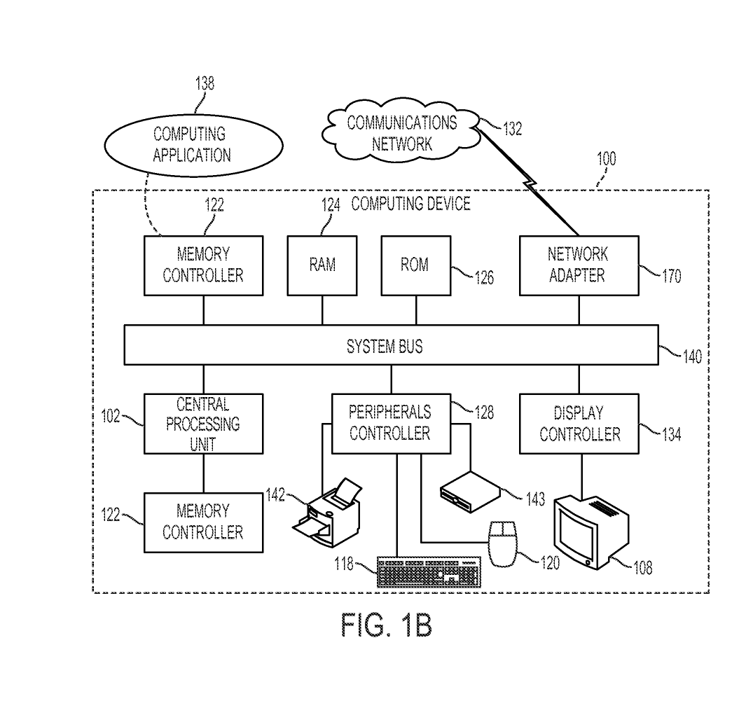

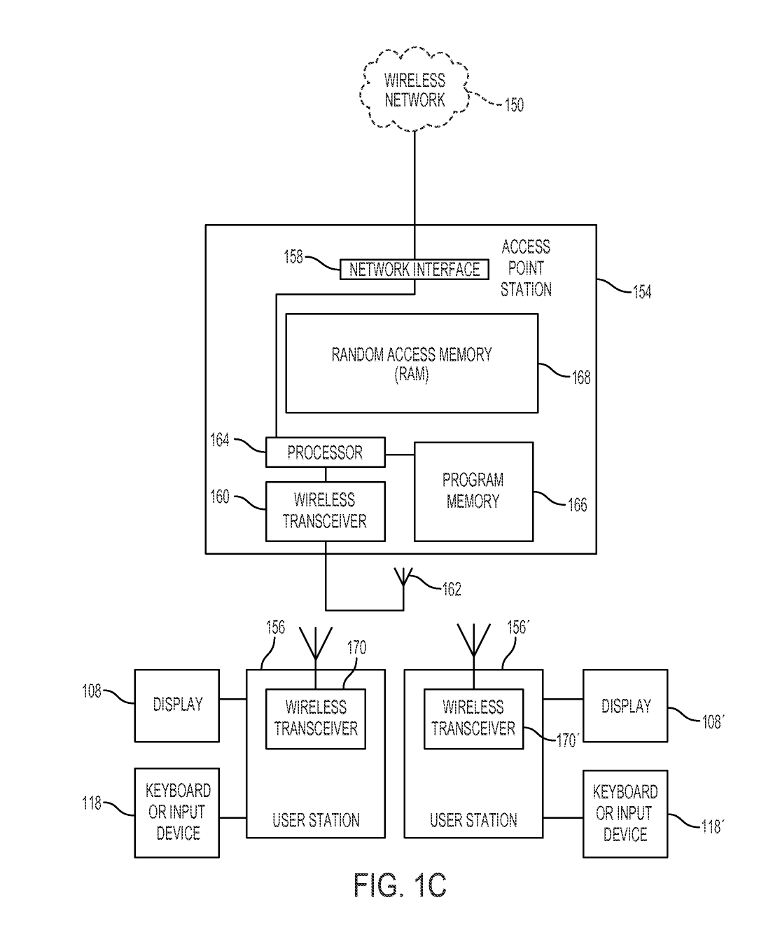

FIG. 1A is a block diagram showing a representative example logic device through which a browser can be accessed to implement the present invention. A computer system (or digital device) 100, which may be understood as a logic apparatus adapted and configured to read instructions from media 114 and/or network port 106, is connectable to a server 110, and has a fixed media 116. The computer system 100 can also be connected to the Internet or an intranet. The system includes central processing unit (CPU) 102, disk drives or flash drives 104, optional input devices, illustrated as keyboard 118 and/or mouse 120 and optional monitor 108. Data communication can be achieved through, for example, communication medium 109 to a server 110 at a local or a remote location. The communication medium 109 can include any suitable means of transmitting and/or receiving data. For example, the communication medium can be a network connection, a wireless connection or an internet connection. It is envisioned that data relating to the present disclosure can be transmitted over such networks or connections. The computer system can be adapted to communicate with a participant and/or a device used by a participant. The computer system is adaptable to communicate with other computers over the Internet, or with computers via a server.

FIG. 1B depicts another exemplary computing system 100. The computing system 100 is capable of executing a variety of computing applications 138, including computing applications, a computing applet, a computing program, or other instructions for operating on computing system 100 to perform at least one function, operation, and/or procedure. Computing system 100 is controllable by computer readable storage media for tangibly storing computer readable instructions, which may be in the form of software. The computer readable storage media adapted to tangibly store computer readable instructions can contain instructions for computing system 100 for storing and accessing the computer readable storage media to read the instructions stored thereon themselves. Such software may be executed within CPU 102 to cause the computing system 100 to perform desired functions. In many known computer servers, workstations and personal computers CPU 102 is implemented by micro-electronic chips CPUs called microprocessors. Optionally, a co-processor, distinct from the main CPU 102, can be provided that performs additional functions or assists the CPU 102. The CPU 102 may be connected to co-processor through an interconnect. One common type of coprocessor is the floating-point coprocessor, also called a numeric or math coprocessor, which is designed to perform numeric calculations faster and better than the general-purpose CPU 102.

As will be appreciated by those skilled in the art, a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Some embodiments may be implemented in one or a combination of hardware, firmware and software. Embodiments may also be implemented as instructions stored on a non-transitory computer-readable storage medium, which may be read and executed by at least one processor to perform the operations described herein. A non-transitory computer-readable storage medium may include any mechanism for storing information in a form readable by a machine (e.g., a computer). For example, a non-transitory computer-readable storage medium may include read-only memory (ROM), random-access memory (RAM), magnetic disk storage media, optical storage media, flash-memory devices, and other non-transitory media.

In operation, the CPU 102 fetches, decodes, and executes instructions, and transfers information to and from other resources via the computer's main data-transfer path, system bus 140. Such a system bus connects the components in the computing system 100 and defines the medium for data exchange. Memory devices coupled to the system bus 140 include random access memory (RAM) 124 and read only memory (ROM) 126. Such memories include circuitry that allows information to be stored and retrieved. The ROMs 126 generally contain stored data that cannot be modified. Data stored in the RAM 124 can be read or changed by CPU 102 or other hardware devices. Access to the RAM 124 and/or ROM 126 may be controlled by memory controller 122. The memory controller 122 may provide an address translation function that translates virtual addresses into physical addresses as instructions are executed.

In addition, the computing system 100 can contain peripherals controller 128 responsible for communicating instructions from the CPU 102 to peripherals, such as, printer 142, keyboard 118, mouse 120, and data storage drive 143. Display 108, which is controlled by a display controller 163, is used to display visual output generated by the computing system 100. Such visual output may include text, graphics, animated graphics, and video. The display controller 134 includes electronic components required to generate a video signal that is sent to display 108. Further, the computing system 100 can contain network adaptor 136 which may be used to connect the computing system 100 to an external communications network 132.

### II. Networks and Internet Protocol

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

### III. Wireless Networks

Wireless networks can incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

In addition, Multiple Interface Devices (MIDs) may be utilized in some wireless networks. MIDs may contain two independent network interfaces, such as a Bluetooth interface and an 802.11 interface, thus allowing the MID to participate on two separate networks as well as to interface with Bluetooth devices. The MID may have an IP address and a common IP (network) name associated with the IP address.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi (Wireless Fidelity) devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

Additionally devices may be capable of near field communication (NFC) which enables devices to establish radio communication with each other by touching them together or brining the devices into close proximity (typically within a few centimeters). Communication may also be possible between an NFC device and an unpowered NFC chip or tag.

Wireless networks can also involve methods and protocols found in, Mobile IP (Internet Protocol) systems, in PCS systems, and in other mobile network systems. With respect to Mobile IP, this involves a standard communications protocol created by the Internet Engineering Task Force (IETF). With Mobile IP, mobile device users can move across networks while maintaining their IP Address assigned once. See Request for Comments (RFC) 3344. NB: RFCs are formal documents of the Internet Engineering Task Force (IETF). Mobile IP enhances Internet Protocol (IP) and adds a mechanism to forward Internet traffic to mobile devices when connecting outside their home network. Mobile IP assigns each mobile node a home address on its home network and a care-of-address (CoA) that identifies the current location of the device within a network and its subnets. When a device is moved to a different network, it receives a new care-of address. A mobility agent on the home network can associate each home address with its care-of address. The mobile node can send the home agent a binding update each time it changes its care-of address using Internet Control Message Protocol (ICMP).

FIG. 1C depicts components that can be employed in system configurations enabling the systems and technical effect of this disclosure, including wireless access points to which client devices communicate. In this regard, FIG. 1C shows a wireless network 150 connected to a wireless local area network (WLAN) 152. The WLAN 152 includes an access point (AP) 154 and a number of user stations 156, 156′. For example, the network 150 can include the Internet or a corporate data processing network. The access point 154 can be a wireless router, and the user stations 156, 156′ can be portable computers, personal desk-top computers, PDAs, portable voice-over-IP telephones and/or other devices. The access point 154 has a network interface 158 linked to the network 150, and a wireless transceiver in communication with the user stations 156, 156′. For example, the wireless transceiver 160 can include an antenna 162 for radio or microwave frequency communication with the user stations 156, 156′. The access point 154 also has a processor 164, a program memory 166, and a random access memory 168. The user station 156 has a wireless transceiver 170 including an antenna 172 for communication with the access point station 154. In a similar fashion, the user station 156′ has a wireless transceiver 170′ and an antenna 172 for communication to the access point 154. By way of example, in some embodiments an authenticator could be employed within such an access point (AP) and/or a supplicant or peer could be employed within a mobile node or user station. Desktop 108 and key board 118 or input devices can also be provided with the user status. Where the devices incorporate NFC technology, touching of electronic devices would allow for transfer of data.

### IV. Computer Network Environment

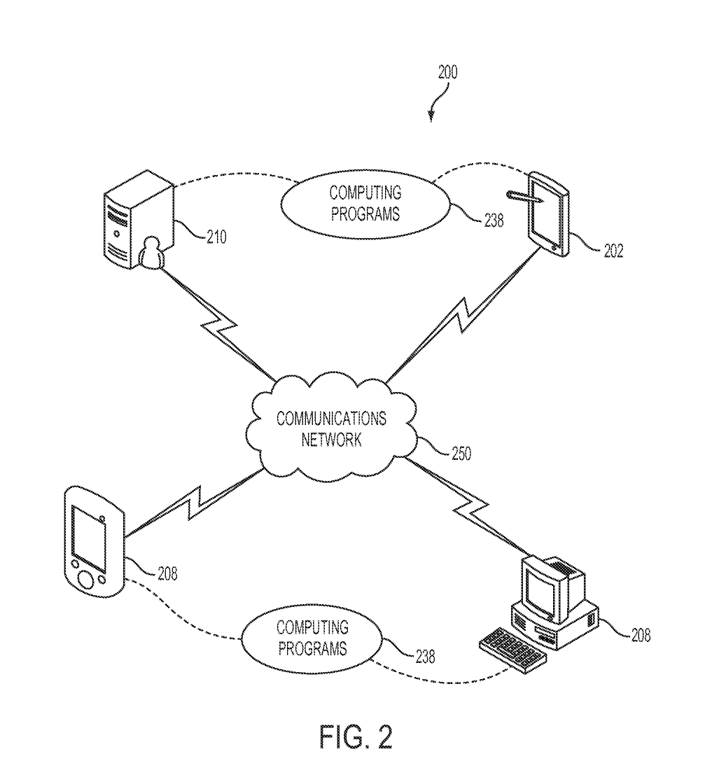

Computing system 100, described above, can be deployed as part of a computer network used to achieve the desired technical effect and transformation. In general, the above description for computing environments applies to both server computers and client computers deployed in a network environment. FIG. 2 illustrates an exemplary illustrative networked computing environment 200, with a server in communication with client computers via a communications network 250. As shown in FIG. 2, server 210 may be interconnected via a communications network 250 (which may be either of, or a combination of a fixed-wire or wireless LAN, WAN, intranet, extranet, peer-to-peer network, virtual private network, the Internet, or other communications network) with a number of client computing environments such as tablet personal computer 202, smart phone 208, personal computer 202, and personal digital assistant. In a network environment in which the communications network 250 is the Internet, for example, server 210 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment 200 can utilize various data security protocols such as secured socket layer (SSL) or pretty good privacy (PGP). Each client computing environment can be equipped with operating system 238 operable to support one or more computing applications, such as a web browser (not shown), or other graphical user interface (not shown), or a mobile desktop environment (not shown) to gain access to server computing environment 200.

In operation, a user (not shown) may interact with a computing application running on a client computing environment to obtain desired data and/or computing applications. The data and/or computing applications may be stored on server computing environment 200 and communicated to cooperating users through client computing environments over exemplary communications network 250. The computing applications, described in more detail below, are used to achieve the desired technical effect and transformation set forth. A participating user may request access to specific data and applications housed in whole or in part on server computing environment 200. These data may be communicated between client computing environments and server computing environments for processing and storage. Server computing environment 200 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### V. Devices for Measuring Peak Flow-Rate which are Configurable to Operate in the Computing and Network Environments to Achieve a Desired Technical Effect or Transformation

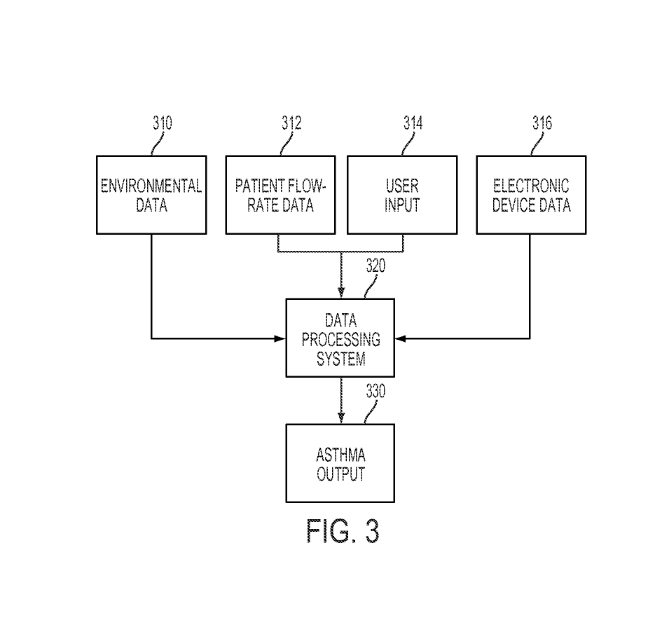

FIG. 3 illustrates the interrelationship between components of a suitable system according to the disclosure. Environmental data 310 can be obtained from a sensor associated with an electronic device, such as those disclosed above in reference to FIG. 2, or can be acquired from a remote source such as a website that provides environmental data based on a location for the electronic device, such as that determined by GPS. Patient (user) flow-rate data 312 is also provided by the flow-rate detector. Additionally, user input 314 can be obtained, if desired, as well as electronic device data 316, such as location, altitude, temperature, etc. The information is then processed, using a data processing system 320 which is located either on a network or on the electronic device, to generate an asthma output 330. The information can then be transmitted back to one or more remote location (such as a physician's office or other users).

The system can analyze the environmental information for a particular patient or user based on one or more of the following: the patient's prior history under similar conditions, the real time results of other system users having a similar profile or a similar history, the historical results of other system users having a similar profile, or a similar history under similar conditions previously experienced.

The system is configurable to send a patient an alert to the potential of a respiratory episode, suggestions for preparing for a change in environment, a change in the patient's risk profile, historical information about reactions to current or predicted future conditions in a specific geography, and so on. Additionally, the system can provide additional data, alerts, or reports to the user's healthcare provider to enable to healthcare provider to monitor conditions and propose changes in treatment protocol, if desired. In some configurations, the system is configurable to alert emergency services to the location of the user and the nature of the respiratory event.

In some configurations the environmental data can be continuously received during operation of an onboard environmental sensor. Environmental sensor data can, for example, be collected by sensors on or near the body of the patient which may include a humidity sensor, a temperature sensor, an altitude sensor, a GPS sensor, and an airborne particle sensor, or other suitable sensor. The sensor can be associated with the device or with another electronic device. In some instances, environmental data can be pre-processed to generate an indication of environmental asthma triggers. The information can also be compared to historical data for that patient as well as historical data from other patients with similar profiles and environmental conditions. In other configurations, the environmental information is available from an external source such as www.pollen.com. In some configurations, altitude is determinable based on the GPS location.

Other aspects include one or more networked devices. The networked devices comprise: a memory; a processor; a communicator; a display; and an apparatus for detecting expiry flow-rate as discussed herein.

Communication systems are configurable to have at least one of an API engine connected to at least one of the electronic device to create a message about respiratory episode data and transmit the message over an API integrated network to a recipient having a predetermined recipient user name, an SMS engine connected to the system to create an SMS message about the respiratory episode data and transmit the SMS message over a network to a recipient device having a predetermined respiratory episode data recipient telephone number, and an email engine connected to the system to create an email message about the respiratory episode data and transmit the email message over the network to a recipient email.

A storing module can also be provided on the server computer system for storing the respiratory episode data on the system for measuring the characteristic of the flow-rate server database. Moreover, the system is connectable to a server computer system over at least one of a mobile phone network and an Internet network, and a browser on the recipient electronic device which can be used to retrieve an interface on the server computer system. Additionally, a plurality of email addresses are held in a system database and fewer than all the email addresses are individually selectable from the computer system, the email message being transmitted to at least one data recipient email having at least one selected email address. In some instances the system is connectable to the server computer system over the Internet, and a browser on the electronic device to retrieve an interface on the server computer system. A plurality of user names are held in the system database and fewer than all the user names are individually selectable from the computer system, enabling a message to be transmitted to at least one respiratory episode data recipient user name via an API.

Other aspects include one or more networked apparatuses. The networked apparatuses comprise: a memory; a processor; a communicator; a display; and an apparatus for receiving user input rates as described herein.

Systems are configurable to incorporate a variety of operating parameters such as those provided for in TABLE 1.

TABLE 1
OPERATING PARAMETERS

| | |
|---|---|
| Patient/User Profile | Diagnostic (Dx) |
| | Clinical Profile |
| | Medication Requirements |
| Clinical Data | PEF |
| | Wheezometry |
| | Symptom Scoring |
| Environmental Data | Weather |
| | Pollen |
| | Data from other populations, geographies |
| Behavioral Data | Temporal Observations |
| | Individual Use of Rescue |
| | Diet |
| | Exercise |
| Learning | Individual |
| | Population |
| | Trends |

### VI. EXAMPLES

### Example 1

A first user reports to the handheld device (e.g., by entering text or interfacing with a device that takes a biological measurement), that there has been a condition change, e.g., a condition change to high risk. The report includes geographic positioning data, such as a GPS tag. The status of the first user is uploaded to a server via a network. A second user reports that there has been a condition change and the second user report is uploaded to a server via a network. An assessment is then made that both the first and second users are located within a geographic area set by the system for that area (e.g., 0.5 mile radius, 1 mile radius, 1.5 mile radius, 2 mile radius, 2.5 mile radius, 3.0 mile radius, 3.5 mile radius, 4.0 mile radius,. 4.5 mile radius, 5.0 mile radius, etc.). Once a geographic link is established between the first and second user a comparison of user profile data is performed to identify other users having similar profiles positioned in the same geographic region. Additionally, an assessment can be made of a historical response by users to similar conditions. An alert is then generated advising the users to be aware that conditions exist that may cause them to have an episode. The alert can also be configured to provide specific suggestions for action by the users. The alert can be sent to the users via email, text message, pop-up, or any other mechanism selected by the user.

### Example 2

A first user reports to the handheld device (e.g., by entering text or interfacing with a device that takes a biological measurement), that there has been a condition change, e.g., a condition change to high risk. The report includes geographic positioning data, such as a GPS tag. The status of the first user is uploaded to a server via a network. A second user reports that there has been a condition change and the second user report is uploaded to a server via a network. An assessment is made that both users are located within a geographic area set by the system (e.g., .5 mile radius, 1 mile radius, 1.5 mile radius, 2 mile radius, 2.5 mile radius, etc.). The system determines that both users are located within a geographic area that is a forest. An assessment is made of current environmental triggers in the geographic area. An assessment is made of users in the network who are, based on GPS positioning, approaching the area. An alert is then generated advising the users to be aware that conditions exist that may cause them to have an episode in the area they are approaching. The alert can also be configured to provide specific suggestions for action by the users or can be in the form of a personalized forecast for the user. The alert can be sent to the users via email, text message, pop-up, or any other mechanism selected by the user.

In some configurations, the alert can compare conditions to a prior incident experienced by the user to give additional context to the user.

### Example 3

A first user's handheld electronic device sends GPS location coordinates to the network via a communication network. Based on the location, data are retrieved about environmental conditions including, but not limited to, local weather, air quality, and pollen count. A report of predicted probability of experiencing a respiratory episode is developed and provided via the communication network to the user. The report can be based on the user's history, the user's profile, the probability of experiencing a problem based on other user's histories, or combinations thereof

At the time of detecting a new location, the system can query the user to determine whether the change is temporary (e.g., a vacation) or permanent (e.g., a relocation). Additionally, for temporary changes, the system can query the length of time and provide information concerning environmental factors impacting respiratory function based on known or historical data.

### Example 4

A first user's handheld electronic device sends GPS location coordinates to the network via a communication network. The system keeps track of the user's history, including geographic location. When the user relocates, permanently or semi-permanently, to a new geographic area, the system sends a medication reminder to the handheld electronic device over the network. If a user relocates to a geographic location where a change in medication might be appropriate a notice can be delivered that identifies current medication and its optimal application, change in environmental factors, and a suggestion that the user visit his or her healthcare practitioner to ensure no change in medication or treatment protocol is appropriate due to the change in circumstances.

### Example 5

A first user provides information associated with the user of a rescue inhaler or other interventional procedures into a program accessible via an electronic device. The device associates a data and time stamp along with GPS data and environmental information from the device or from third party sources. The information is analyzed to identify potential triggers for the patient. When the system detects that the user is in conditions approaching those associated with an earlier incident, an alert is generated to facilitate the patient's ability to take evasive behavioral steps to avoid or minimize the likelihood of a respiratory episode.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system comprising:
an electronic device configurable to be in communication with a communication network;
a computer executable instruction that, when executed by a processor determines a likelihood of a respiratory event for a user based on two or more of historical user data, current environmental data, current physiological data for one or more other users in a similar geographic location, and historical data for one or more of the other user in a similar geographic location.

2. The system of claim 1 wherein one or more of the historical user data and historical data for one or more other users is accessible via the communication network.

3. The system of claim 1 further comprising a messaging system wherein the messaging system is capable of delivering a respiratory event message to one or more of each of the user, one or more of the other users, and a healthcare provider.

4. The system of claim 1 wherein the electronic device is at least one of a respiratory sensor and a mobile communication device.

5. The system of claim 1 wherein the computer executable instruction is processed on one or more of the electronic device, a remote server, and an electronic device within near field communication range.

6. A non-transitory computer readable medium storing instructions that, when executed by a computing device, causes the computing device to perform a method, the method comprising:
receiving one or more of GPS location and a physiological condition indication for a first user;
at least one or more of analyzing, monitoring, evaluating, and providing a respiratory prediction for a second user based on the GPS location of the second user and the physiological condition indication for the first user.

7. The non-transitory computer readable medium of claim 6 further comprising the step of at least one or more of analyzing and evaluating one or more of a historical data for the first user and historical data for second user.

8. The non-transitory computer readable medium of claim 6 wherein the medium is capable of generating and delivering a respiratory event message to one or more of the user, one or more of the other users, and a healthcare provider.

9. The non-transitory computer readable medium of claim 6 wherein the computing device performing the method is at least one of a respiratory sensor and a mobile communication device.

10. The non-transitory computer readable medium of claim 6 wherein the method is processed on one or more of the electronic device, a remote server, and an electronic device within near field communication range.

11. A computing device comprising:
a processor configured to:
receive one or more of GPS location and physiological condition indication for a first user;
at least one or more of analyze, monitor, evaluate, and provide a respiratory prediction for one or more second users based on the GPS location of the one or more second users and the physiological condition indication for the first user.

12. The computing device of claim 11 wherein the processor is configured to at least one or more of analyze and evaluate one or more of the historical patient data and historical data for one or more of the first user and the one or more second users.

13. The computing device of claim 11 wherein the processor is capable of generating and delivering a respiratory event message to one or more of the first user, and one or more of the second users, and a healthcare provider.

14. The computing device of claim 11 wherein the processor is associated with an electronic device and the electronic device is at least one of a respiratory sensor and a mobile communication device.

15. The computing device of claim 14 wherein the processor is in communication with one or more of the electronic device, a remote server, and an electronic device within near field communication range.

16. A method comprising:
receiving one or more of GPS location and a physiological condition indication for a first user;
at least one or more of analyzing, monitoring, evaluating, and providing a respiratory prediction for one or more second users based on the GPS location of the one or more second users and the physiological condition indication for the first user.

17. The method of claim 16 further comprising the step of at least one or more of analyzing and evaluating one or more of a historical data for the first user and historical data for second user.

18. The method of claim 16 further comprising the step of generating and delivering a respiratory event message to the first user.

19. The method of claim 16 further comprising the step of generating and delivering a respiratory event message to one or more of the other users.

20. The method of claim 16 further comprising the step of generating and delivering a respiratory event message to a healthcare provider or a central location.
